(19) United States
(12) Patent Application Publication
(10) Pub. No.: US 2025/0288204 A1
(43) Pub. Date: Sep. 18, 2025

(54) REMOTE DENTAL HEALTHCARE

(71) Applicant: Dentlytec G.P.L. LTD., Hod Hasharon (IL)

(72) Inventors: Benny PESACH, Rosh Haayin (IL); Amitai REUVENNY, Kfar-Saba (IL)

(73) Assignee: Dentlytec G.P.L. LTD., Hod Hasharon (IL)

(21) Appl. No.: 18/860,149

(22) PCT Filed: Apr. 27, 2023
(86) PCT No.: PCT/IL2023/050429

§ 371 Date: Oct. 25, 2024

Related U.S. Application Data
(60) Provisional application No. 63/336,175, filed on Apr. 28, 2022.

(51) Int. Cl. A61B 5/00; G16H 10/20; G16H 80/00

(52) U.S. Cl. CPC A61B 5/0013; A61B 5/4547; A61B 5/4836; G16H 10/20; G16H 80/00

(57) ABSTRACT

Systems and methods for providing, monitoring, and managing dental telehealth are described. In some embodiments, user collected data (self-collected data) are obtained (e.g., in image form) and integrated (optionally along with other patient-specific information) into a decision-supporting data system. In some embodiments, user-collected images are obtained using a camera of a personal electronic device equipped with an optical add-on that assists in transferring the field of view of the camera to an offset location, e.g., within the mouth. The data system optionally further takes into account data aspects of a dental health system such as individual procedure costs, overall treatment costs, treatments, availabilities, performance levels, and outcomes. Data characterizing any of these may in turn be taken into account. Decision making is thereby optionally promoted for any of several stakeholders such as insurance provides, health care organizations, individual health care providers, and/or assessors thereof.

### RELATED APPLICATIONS

This application claims the benefit of priority of U.S. Provisional Patent Application No. 63/336,175 filed on 28 Apr. 2022, the contents of which are incorporated herein by reference in their entirety.

### FIELD AND BACKGROUND OF THE DISCLOSURE

Embodiments of the present disclosure relate to remote healthcare, and more particularly, but not exclusively, to remote dental healthcare.

Background art includes:

International Patent Application Publication No. WO2019/102480, which discloses “A dental device tracking method including acquiring, using an imager of a dental device, at least a first image which includes an image of at least one user body portion outside of a user's oral cavity; identifying the at least one user body portion in the first image; and determining, using the at least the first image, a position of the dental device with respect to the at least one user body portion.”

International Patent Application Publication No. WO2021/224929, which discloses “A dental add-on for an electronic communication device having a screen and an imager, the add-on including: a body comprising: a distal portion sized and shaped to be at least partially inserted into a human mouth within, in one or more dimension, one or both dental arches; and an optical path extending from an optical element of the electronic communication device, through the body to the distal portion and configured to adapt a FOV of the optical element for dental imaging; a connector for connection of the add-on body to the electronic communication device.”

### SUMMARY OF THE INVENTION

According to an aspect of some embodiments of the present disclosure, there is provided a method of dental telehealth provision including: receiving patient self-input data, provided digitally, using a home dental camera; identifying a dental health issue from the patient self-input data; generating a treatment protocol including at least one activity to be carried out by the patient for treatment of the dental health issue; and communicating the treatment protocol to the patient.

According to some embodiments of the present disclosure, the home dental camera includes a camera of a personal electronic device coupled to an add-on which spatially transfers a field of view of the camera through a body of the add-on.

According to some embodiments of the present disclosure, the home dental camera is hosted by (e.g., connected to) a toothbrush device.

According to some embodiments of the present disclosure, generating includes a dental healthcare professional specifying one or more feature of the treatment protocol.

According to some embodiments of the present disclosure, generating includes using a look-up table.

According to some embodiments of the present disclosure, generating includes using a best practices database.

According to some embodiments of the present disclosure, the method includes adjusting the treatment protocol based on one or more of user preference and user likely compliance.

According to some embodiments of the present disclosure, the treatment protocol includes instructions for one or more of: using medication; and dental self-treatment.

According to some embodiments of the present disclosure, the self-input data includes at least one image.

According to some embodiments of the present disclosure, the self-input data includes a plurality of images, where, in some embodiments, at least two of the plurality of images including different portions of a user's mouth.

According to some embodiments of the present disclosure, the self-input data includes a plurality of images acquired during different scans.

According to some embodiments of the present disclosure, identifying includes displaying the user self-input to a healthcare professional; and receiving inputs from the healthcare professional.

According to some embodiments of the present disclosure, the identifying includes using artificial intelligence to automatically identify the dental health issue.

According to some embodiments of the present disclosure, the self-input data includes a plurality of images acquired during different scans over a long period; and the identifying includes identifying variation in the oral cavity over the long period.

According to some embodiments of the present disclosure, the identifying includes categorizing the dental health issue; and wherein the generating includes generating based on the categorizing.

According to some embodiments of the present disclosure, the categorizing includes categorizing the dental health issue as one or more of; acute, chronic, or a potential future dental health issue.

According to some embodiments of the present disclosure, the method includes monitoring the user.

According to some embodiments of the present disclosure, monitoring includes receiving additional patient self-input data, provided digitally, using a home dental camera.

According to some embodiments of the present disclosure, monitoring includes communicating monitoring instructions to the user.

According to some embodiments of the present disclosure, the method includes assessing outcome of the treatment protocol using the additional patient self-input data.

According to some embodiments of the present disclosure, the method includes adjusting the treatment protocol based on the assessing.

According to an aspect of some embodiments of the present disclosure, there is provided a method of dental telehealth provision including: receiving patient self-input data, provided digitally, using a home dental camera; analyze the self-input data to: identify a dental health issue; select a healthcare professional from a plurality of healthcare professionals, based on a rating of the healthcare professional for treatment of the dental health issue.

According to some embodiments of the present disclosure, the method includes treating of the patient by the healthcare professional; and monitoring the patient by receiving additional patient self-input data.

According to some embodiments of the present disclosure, the method includes adjusting the rating, based on the monitoring.

According to some embodiments of the present disclosure, the rating includes one or more of a cost parameter, a likelihood of successful treatment; a likelihood of needing additional treatment, and a reported parameter of painfulness of procedure.

According to an aspect of some embodiments of the present disclosure, there is provided a method of dental telehealth provision including: receiving patient self-input data for a plurality of patients; receiving individual patient self-input data for an individual patient; determining an individual patient health risk profile using the patient self-input data and the individual patient self-input data; generating, for the individual patient, using the individual patient health risk profile the patient self-input data, a projected cost of dental health treatment over an insurance time period.

According to some embodiments of the present disclosure, the method includes assessing the self-input data for the plurality of patients.

According to some embodiments of the present disclosure, the assessing includes receiving input from a dental healthcare professional.

According to some embodiments of the present disclosure, the assessing includes assessing using artificial intelligence.

According to some embodiments of the present disclosure, the determining includes using a risk calculation table.

According to some embodiments of the present disclosure, the determining includes using machine learning with the patient self-input data as an input.

According to some embodiments of the present disclosure, the determining takes into account one or more of; patient demographics, results of a check-up, a patient compliance level.

According to some embodiments of the present disclosure, the determining includes implementing one or more mitigating protocol; wherein the generating is based on the mitigating protocol.

According to some embodiments of the present disclosure, the mitigating protocol includes one or more of a treatment protocol, a monitoring protocol, and an education protocol.

According to some embodiments of the present disclosure, the determining includes assessing individual user input-data as to compliance with the one or more mitigating protocol; wherein the generating is based on the compliance with the one or more mitigating protocol.

According to some embodiments of the present disclosure, the method includes communicating one or more feature of the individual patient health risk profile to the individual patient.

According to an aspect of some embodiments of the present disclosure, there is provided a method of dental telehealth provision including: receiving patient treatment data; monitoring treatment of the patient including receiving patient self-input data; providing feedback as to efficacy of a treatment, based on the monitoring.

According to some embodiments of the present disclosure, the patient treatment data includes a protocol for home treatment.

According to some embodiments of the present disclosure, the providing includes adjusting the protocol, based on the monitoring.

According to an aspect of some embodiments of the present disclosure, there is provided a method of group dental care provision including: receiving patient self-input data, over time, for a plurality of patients and an assessment thereof, receiving dental healthcare professional resource data; determining, based on the resource data, the self-input data, and the assessment an optimized treatment plan including: treatment type for each of the plurality of patients; and a treatment schedule for the plurality of patients.

According to some embodiments of the present disclosure, the dental healthcare professional resource data includes time availability for one or more dental healthcare professional.

According to some embodiments of the present disclosure, the treatment plan is optimized to be minimal cost, for a time period.

According to some embodiments of the present disclosure, the determining of the treatment plan includes using projected cost per patient for each treatment type.

According to some embodiments of the present disclosure, the determining of the treatment plan includes using the resources requirement and the dental healthcare professional resource data.

According to some embodiments of the present disclosure, the assessment includes urgency of treatment.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A is a simplified schematic of a system 100, according to some embodiments of the present disclosure;

FIG. 1B is a simplified schematic of an electronic device, according to some embodiments of the present disclosure;

FIG. 2 is a simplified schematic of a dental device, according to some embodiments of the present disclosure;

FIG. 3 is a simplified schematic of an add-on coupled to an electronic device, according to some embodiments of the present disclosure;

FIG. 4 is a flow chart of user experience with and/or as part of some embodiments of the present disclosure;

FIG. 5 is a flow chart illustrating healthcare professional interaction with and/or as part of some embodiments of the present disclosure;

FIG. 6 is a flow chart illustrating assessor use of healthcare provider and/or healthcare professional data, according to some embodiments of the present disclosure;

FIG. 7 is a flow chart illustrating use of user data and healthcare professional data by a third party, according to some embodiments of the present disclosure;

FIG. 8 is a flow chart illustrating use of collective data, according to some embodiments of the present disclosure;

FIG. 9 is a simplified block diagram illustrating recommendation and/or selection generation, according to some embodiments of the present disclosure;

FIG. 10 is a simplified schematic block diagram illustrating protocol generation, according to some embodiments of the present disclosure; and

FIG. 11 is a flow chart illustrating an exemplary use case, according to some embodiments of the present disclosure.

### DESCRIPTION OF SPECIFIC EMBODIMENTS OF THE INVENTION

Some embodiments of the disclosure relates to remote healthcare, and more particularly, but not exclusively, to remote dental healthcare.

### Overview

A broad aspect of some embodiments of the disclosure relates to tailoring of healthcare to an individual.

An aspect of some embodiments of the disclosure relates to determining individual patient parameters, and using these parameters to determine features of treatment that the patient receives. In some embodiments, the parameters include the individual's current state of health (e.g. one or more specific healthcare issues), and/or one or more factors such as how likely the individual is to comply with instructions (e.g. instructions for treatment and/or maintenance of health and/or aftercare to a treatment).

In some embodiments, patient parameters for an individual patient, are determined using individual data from the specific patient, and collective data from a plurality of patients and/or healthcare professionals.

In some embodiments, one or both of the individual data and the collective data are acquired by scanning. In some embodiments, scanning is by a user (or a user caregiver, e.g. in a home setting) with a scanner including one or more imagers, and/or using a personal electronic device. In some embodiments, the scanner is a dental device e.g. a toothbrush. In some embodiments, the personal electronic device is a smartphone to which is attached, in some embodiments, a scanner. The personal electronic device is configured, for example by the use of add-on optical elements, to convert the field of view (FOV) of one or more optical elements (e.g. imagers) of the original device to be defined relative to a new point of view (POV). The new point of view is established by an optical aperture which is offset and/or re-oriented relative to the POV of the original device.

For example, the add-on optical elements include, in some embodiments, a periscope sized and/or shaped for insertion into the oral cavity. In some embodiments, the scanner is an intraoral scanner (optionally, a low-cost intraoral scanner) that provides a 3-D model of the oral cavity. In some embodiments, the scanner provides an image of the oral cavity. In some embodiments the 3D model is used to create custom clear aligners and/or custom remineralization and fluoride trays and/or custom teeth whitening trays and/or custom mouth guards for tooth abrasion treatment and/or custom oral device for sleep apnea treatment and/or other custom 3D devices that require the unique 3D model of the patient oral cavity to produce a device that will be attached to the patient oral element like teeth or gums from different materials (for example soft ethylene-vinyl acetate and/or polymethyl methacrylate and/or trimethyl orthosilicate).

In some embodiments, the collective data is used to generate one or more predictions. For example, the historical data is used to provide prediction and/or risk assessment of one or more future patient parameters. The prediction may be, for example, one or more of: future dental health feature(s), predication of efficacy of different treatment(s) (e.g. for one or more healthcare issues), and prediction of cost of dental healthcare for the individual.

In some embodiments, suggestions based on the predicted patient parameter(s) relate to one or more of: a treatment type, which dental healthcare professional of a plurality of professionals is to provide the treatment, feature(s) of monitoring and/or aftercare for the individual, and insurance premiums for an individual patient.

In some embodiments, a plurality of suggestion options are provided with estimates of different parameters for each option. For example, with reference to treatment types: in some embodiments, for each potential treatment (e.g. of a particular healthcare issue) one or more parameters is determined. The parameters optionally include, For example, one or more of: a short and/or long term and/or total cost of the treatment (e.g. where some treatments have a high initial cost, but lower risk of additional treatment being necessary compared to a treatment with a low initial cost but a higher risk of additional costs); a likelihood of success of a treatment (for example, a subject who is highly compliant may have a higher likelihood of success of a home-based treatment than a subject who is less compliant); and a speed of the treatment.

In some embodiments, risk is presented to the patient; for example, details as to risks of future events are presented to the patient. Optionally, risk is presented together with actions the patient can take to reduce their risk.

In some embodiments, patient risk is determined, including risk for various dental healthcare outcomes, including variance of the risk for the individual and/or groups of individuals.

In some embodiments, monitoring provides the patient with more frequent access to dental healthcare. This has potential advantages for providing early detection of dental healthcare issues (for example tooth decay, cavities, de-mineralization, gingivitis, periodontitis, tooth abrasion and/or tooth discoloration), and/or for reducing the “surprise factor” of diagnoses based on in-person monitoring (which may be less frequent in comparison to monitoring e.g. as described herein).

A broad aspect of some embodiments of the disclosure relates to using collective data, including, for example, user self-provided data, to provide potential advantages, for example, cost reduction and/or increases to patient care level.

In some embodiments, insurance premiums for individuals are determined using the individual's data, and collective data from a plurality of individuals. Where, for example, premiums are adjusted based on one or more of assessment of current dental health, and future dental health, where future dental health, in some embodiments, is predicted using likelihood of compliance of the individual.

In some embodiments, collective data regarding a plurality of dental professionals, for example, including cost of procedure(s), efficacy of procedure(s) and/or available resources of the dental professionals is used to determine, for individuals and/or treatment type(s), a recommendation as to which dental professional should perform a function (e.g. one or more of monitoring, diagnosis, and treatment).

In some embodiments, different professionals are used for different tasks. A potential advantage of this is to decouple diagnosis/treatment and associated financial incentives (e.g. potentially resulting in lower costs of treatment overall).

A broad aspect of some embodiments of the disclosure relates to using periodic monitoring of individuals, wherein monitoring data is provided by the individual (or caregiver to the individual, e.g. in a home setting) to provide increased quality dental healthcare.

In some embodiments, monitoring includes scanning of dental features. Optionally, the dental feature in turn is related to one or more features of treatment, for example as described for the previous aspect.

In some embodiments, monitoring is used to provide early identification of health issues and/or early treatment (e.g. preventative treatment). This has the potential advantage of reducing cost and/or severity of dental healthcare issues. In some embodiments, early identification is of cavities, the early identification having a potential advantage of reducing an invasiveness of the filling procedure and/or treatment cost. In some embodiments, early identification is of oral cancer, with a potential advantage of improving patient outcome. In some embodiments, early identification is of gum disease; for example, identification prior to damage resulting from the gum disease.

In some embodiments, monitoring is used to provide feedback as to opportunities to improve dental aesthetics (For example during tooth whitening protocol or teeth alignment). In some embodiments, monitoring is done during the at-home treatments, for example while using custom whitening trays of custom clear aligners).

In some embodiments, monitoring is used to assess effectiveness of a current treatment. For example, the monitored treatment is home treatment in some embodiments (i.e., a treatment not performed by a dental professional and/or not performed in a dental surgery). Monitoring may be used to assess whether the treatment is one or both of executed correctly and effective (For example to track tooth abrasion reduction when using generic and/or custom mouth guards, to verify remineralization when using generic and/or custom remineralization and/or fluoride trays, to monitor gum diseases treatment when using generic and/or custom periodontal medications trays). Information from the monitoring, in some embodiments, enables an early change in treatment if the initial treatment is not effective; for example, a switch from home treatment to a treatment provided by a healthcare professional.

In another example, monitoring provides after-care for a patient after a procedure (e.g. a procedure performed by a dental healthcare professional). Potentially, home-monitoring provides early diagnosis of post-procedure issues and/or reduces a need for in-person aftercare.

In some embodiments, monitoring comprises remote monitoring. In some embodiments, the remote monitoring is of treatment (for example, follow-up treatment after a procedure) in a home setting. This potentially reduces a need for in-person interaction between healthcare professional(s) and subjects. For example, a frequency of visits and/or durations of visits is reduced.

In some embodiments, user self-scan frequency is more than once a year; for example: every 6 months, or every 1-3 months, or every month, or weekly, or daily (e.g. when scanning is using a toothbrush). Optionally scanning is at a lower or higher or intermediate frequency or range. In some embodiments, user self-scan frequency is defined by frequency of in-person check-ups. For example, the user provides 1-20 scans between check-ups; or a lower, higher or intermediate range or number of scans.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of inventions disclosed therein, are capable of other embodiments or of being practiced or carried out in various ways.

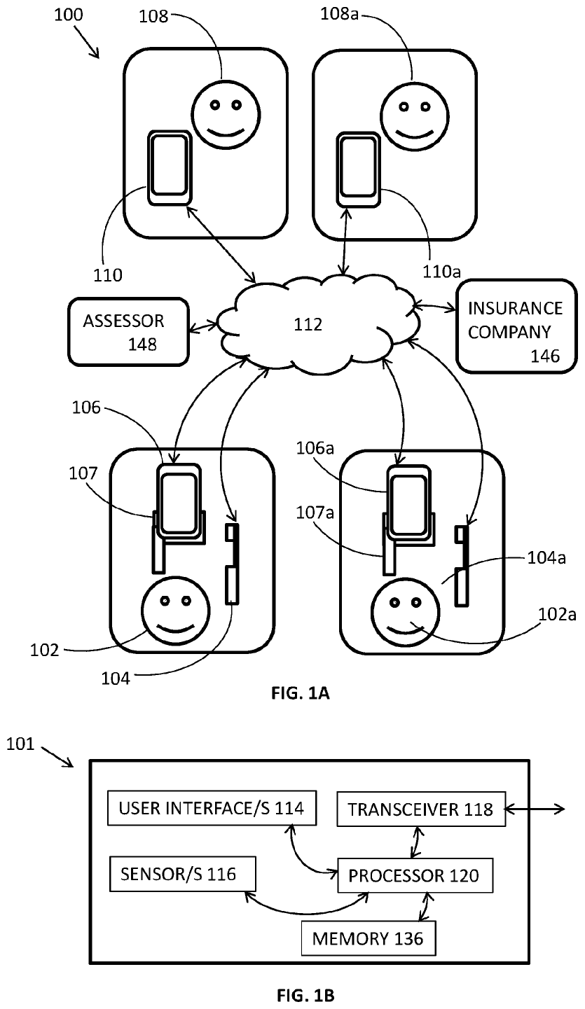

FIG. 1A is a simplified schematic of a system, according to some embodiments of the disclosure.

In some embodiments, monitoring is of one or more users 102, and 102a. In some embodiments, user data is collected using one or more of electronic devices 104, 106, 104a, and 106a.

In some embodiments, the user 102, 102a collects data from themself. Alternatively or additionally, in some embodiments, a care giver (e.g. parent and/or home and/or institutional caregiver) collects data.

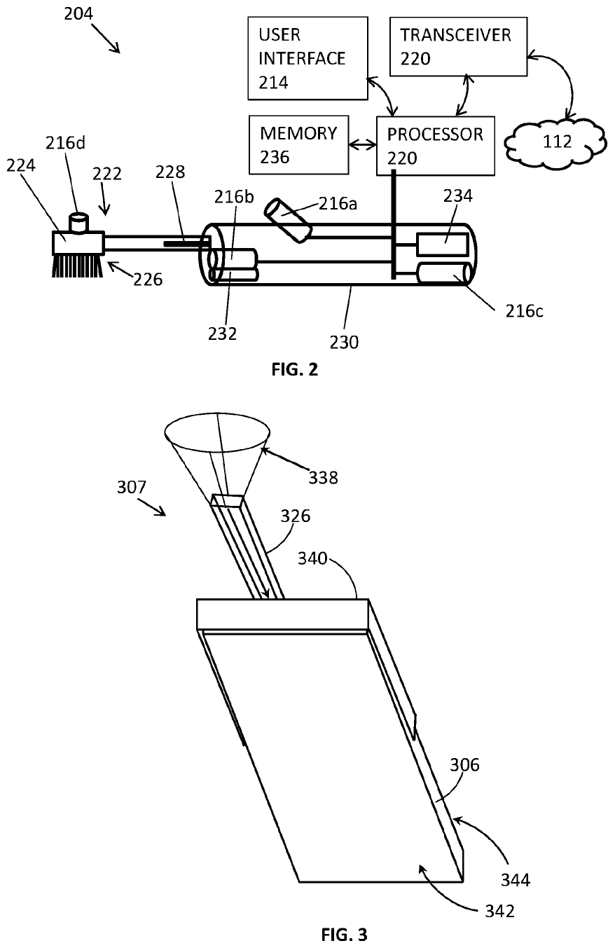

In some embodiments, user data is collected using a toothbrush device 104, 104a which includes one or more sensors and/or user inputs for acquisition of user data. Toothbrush device 104, 104a, in some embodiments, optionally includes one or more features as described in relation to toothbrush device 204 of FIG. 2.

In some embodiments, user data is collected using one or more sensors and/or user interfaces of a personal electronic device 106, 106a (e.g. a smartphone). For example, user data is acquired using electronic device 106, 106a coupled to an adaptor 107, 107a. In some embodiments, adaptor 107, 107a includes a portion sized and/or shaped for inserting into the user's mouth. In some embodiments, adaptor 107, 107a includes one or more features as described in relation to adaptor 307 of FIG. 3.

In some embodiments, a user purchases a self-scanning device and/or pays for use of the device and/or access to the system which receives monitoring data. In some embodiments, e.g. in return for the user's financial investment and/or time investment of self-scanning and/or inputting data into system 100, the user receives lowered premiums; optionally adjusted based on the user compliance with scanning and/or other instructions.

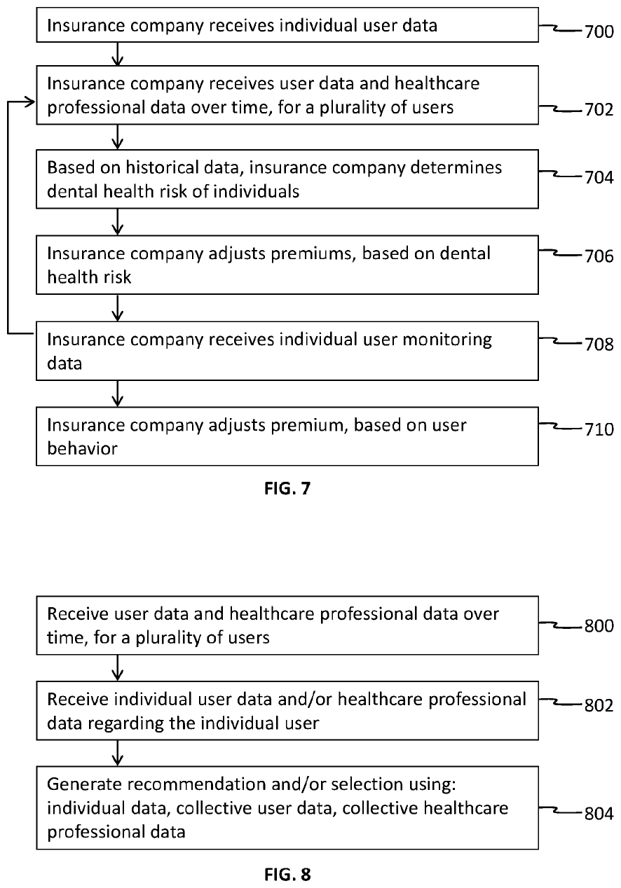

In some embodiments, user data is shared externally 112, for example, for processing. Optionally, the processing includes one or more features, for example as described in relation to FIG. 7.

In some embodiments, data processing cloud 112 and/or a processing application hosted by one or more portions of system 100 uses artificial intelligence (AI) to analyze user data (e.g. one or more images) to automatically detect (e.g. to diagnose) potential dental healthcare problems.

In some embodiments, users 102, and/or 102a (and/or their caregivers) receive communications; for example instructions and/or responses to questions. Communications are accessed, for example, via one or more user interfaces. In some embodiments, the user interface is provided by toothbrush device 104, 104a. In some embodiments, the user interface is provided by personal electronic device 107, 107a. Optionally, another user interface is used; e.g. a device accessing an internet-accessible portal.

In some embodiments, system 100 includes one or more insurance companies 146. In some embodiments, system 100 includes one or more assessors 148.

In some embodiments, data is communicated to a healthcare professional 108, 108a; and/or the healthcare professional inputs data through one or more electronic devices 110, 110a.

In some embodiments, healthcare professional 108, 108a provides an assessment and/or diagnosis for the user, based on data communicated to the healthcare professional 108, 108a. A potential advantage of including in particular a plurality of healthcare professionals 108, 108a within system 100 is the ability to de-couple diagnosis from treatment. For example: in some embodiments, a first healthcare professional provides diagnosis but a different healthcare professional provides the treatment. In some embodiments, a user selects healthcare professional(s). Optionally the insurance company (e.g. private insurance company, state-mandated health service organization or governmental public health services) selects healthcare professional(s) who perform for different tasks.

In some embodiments, the diagnosing professional is selected based on time availability and/or cost, optionally independent of the physical location of the healthcare professional. Additionally or alternatively a treatment professional is selected based on their physical location; for example if the treatment begins in-person, or begins or remotely but includes an element provided in-person.

In some embodiments, selection of a healthcare professional is based on data acquired from a plurality of users and/or a plurality of healthcare professionals e.g. over time. For example, for a particular treatment and/or other relevant user characteristic (e.g. likelihood of compliance) the healthcare professional is selected. Criteria for selection may include, for example, cost, historical proficiency and/or outcome history. In some embodiments, the user selects the healthcare professional.

In some embodiments, insurance company 146 selects the healthcare professional. Criteria for selection may include, for example, reduced final cost of the treatment and/or collective costs for a plurality of users requiring the treatment.

Consideration of final/collective costs may, for example, result in selection of a more expensive professional for initial work; e.g., insofar as a risk of re-occurrence and/or need for further treatment is reduced with this professional.

In some embodiments, a possessor generates a healthcare treatment type recommendation; for example, to reduce collective cost of treating a group of people requiring this treatment and/or to increase a speed of conclusion of treatment.

In some embodiments, data from a plurality of users, for example, data acquired over time, is used to generate recommendations provided to individual healthcare professionals; e.g. recommendations as to which users to treat and/or which type(s) of treatment to specialize in (and/or devote more resources to). Recommendations are be based, for example, on demand and/or potential profit.

In some embodiments, one or more users 102, 102a communicates with and/or agrees to use of their user data by one or more other entities; e.g. one or more healthcare practitioners 108, 108a, insurance company 146 and/or assessor 148.

In an example scenario, a dental health practitioner guarantees a treatment, the guarantee being based on the user following one or more protocols, the monitoring of which is, for example, provided by the user (and/or user caregiver). In another example scenario, user 102, 102a, receives insurance provided by insurance company 146. The premium and/or details of insurance coverage are affected by an extent to which the user follows protocols. The monitoring of the protocols is, for example, provided by the user (and/or user caregiver).

In some embodiments, an insurance company 146 selects and/or recommends a particular dental healthcare practitioner, e.g. from among a plurality of practitioners 108, 108a. The selection and/or recommendation is based on one or more criteria; for example, a determined lowest cost, fastest recovery and/or highest chance of recovery without relapse for the particular treatment required.

In some embodiments, acquired data is analyzed by an assessor 148. Based on the data, the assessor provides an assessment of dental healthcare professionals and/or of insurance company 146. In some embodiments, assessor 148 receives data from a plurality of insurance companies, and provides assessments for used in decision making by users selecting which insurance company and/or healthcare professional to use.

FIG. 1B is a simplified schematic of an electronic device 101, according to some embodiments of the disclosure.

In some embodiments, electronic device 101 illustrates one or more of user electronic devices 106, 106a, 110, 110a of FIG. 1A.

In some embodiments, electronic device 110 illustrates one or more dental professional electronic devices 110, 110a of FIG. 1A.

In some embodiments, electronic device 101 includes one or more sensor 116. For example, in an example where electronic device 101 is a user device 106, 106a, 110, 110a; sensors include an optical sensor for user self-scanning of dental features.

In some embodiments, electronic device 101 includes at least one user interface 114. User interface 114 includes, for example one or more optical, visual, haptic input and/or output devices. For example, user interface 114 includes a screen (e.g. touch screen), one or more lights, one or more actuators (e.g. for vibration output), one or more microphones, and/or one or more speakers.

In some embodiments, electronic device 101 is a user-operated device, with transceiver 118 configured for sending and/or receiving data. The data includes, for example, user measurement data, user inputted data, and/or communications for the user (e.g. treatment and/or monitoring and/or education communications).

In some embodiments, electronic device 101 is a dental professional device 110, 110a. Transceiver 118 receives user data (e.g. measurements and/or inputted data) and/or information generated for example as described in relation to FIG. 5. The user data and/or information are displayed to and/or accessed by healthcare professional 108, 108a.

In some embodiments, transceiver 118, transmits data inputted by a healthcare professional, and/or recommendations for the user. Data are recorded in a memory 112.

In some embodiments, electronic device 101 includes a processor 120. Processor 120, in some embodiments, performs a portion of processing functions e.g. as described elsewhere in this document; for example in relation to FIG. 7.

In some embodiments, electronic device 101 includes memory 136. Memory 136 stores data acquired, e.g. from sensors 116 and/or user interfaces 114 through processor 120, and/or from processor 120 itself. In some embodiments, memory 136 stores data received from transceiver 118 (e.g., via operations of processor 120).

FIG. 2 is a simplified schematic of a dental device 204, according to some embodiments of the disclosure.

In some embodiments, dental device 204 includes one or more feature of dental devices as described and/or illustrated in International Patent Application Publication No. WO2019/102480 which is herein incorporated by reference in its entirety.

In some embodiments, dental device 204 includes an elongate element 222, sized and/or shaped for at least partial insertion into a user's mouth. In some embodiments, the elongate element is in the range of 5-20 cm long; for example, about 8 cm long, about 10 cm long, or about 12 cm long. In some embodiments the elongate element is in the range of 0.1-3 cm wide, in the range of 0.1-1 cm wide, or another range.

In some embodiments, elongate element 222 includes a head 224 disposed at a distal end of elongate element 222. Optionally, head 224 is a toothbrush head 224 comprising brushing bristles 226.

In some embodiments, elongate element 222 is coupled to a handle 230, sized and/or shaped to be grasped by a user while inserting head 224 into the user's mouth.

In some embodiments, handle 230 is sized and/or shaped to be comfortably grasped in a user's hand e.g. in a palmar grasp. For example, in some embodiments, handle 230 is elongate. In some embodiments, a length (e.g. central long axis length) of handle 230 is 5-30 cm, 10-20 cm, or within another range of sizes. In some embodiments, an average and/or maximum width measured perpendicular to the central long axis of the handle 230 is 0.5-3 cm or 1-5 cm, or within another range of maximum widths.

In some embodiments, dental device 204 includes one or more sensors 216a, 216b, 216c, 216d. The sensors, in some embodiments, include at least one imager. For example, dental device 204 includes an imager 216a (optionally hosted by handle 230) having a FOV directed away from a POV defined by handle 230 and towards a user; e.g., oriented to capture one or more portions of a user's face and/or jaw. In some embodiments, dental device 204 includes an imager 216b (optionally hosted by handle 230) where the FOV is directed away from a POV defined by handle 230 and towards a user to acquire images including portions within the oral cavity and/or portions of the user's face and/or jaw.

In some embodiments, dental device 204 includes an imager 216c (optionally hosted by handle 230) which is directed away from the user for acquiring image(s) of the user within a mirror opposite the user. In some embodiments, dental device 204 includes an imager 216d (optionally hosted by head 224) which acquires images of the oral cavity; for example, of an area adjacent to bristles 226 when brushing and/or of dental features opposite those being currently brushed (i.e., dental features of an opposing, second jaw are imaged while brushing a first jaw).

In some embodiments, dental device 204 includes one or more illuminators 232. Illuminators 232 optionally illuminate with structured and/or patterned light. In some embodiments, one or more of imagers 216a-216d has an associated illuminator, wherein the illuminator has a field of illumination (FOI) which overlaps with the FOV of the associated imager.

In some embodiments, dental device 204 includes a power supply 234 which supplies power to electronic devices of the dental device; for example, its imager(s), illuminator(s), and/or one or more actuators. The actuator(s) may be configured to move some portion of head 224; for example, actuators for electric toothbrush movement of bristles 226.

In some embodiments, dental device 204 includes one or more user interfaces 214, including one or more features for example as described in relation to user interface 114 of FIG. 1B.

In some embodiments, dental device 204 includes a processor 220. Processor 220 optionally is used, for example, in the implementation of one or more user interface features, for example as described in relation to user interface 114 of FIG. 1B.

In some embodiments, dental device 204 includes a memory 236. Memory 236 optionally is used, for example, in the implementation of one or more user interface features; for example as described in relation to user interface 114 of FIG. 1B.

In some embodiments, dental device 204 includes a transceiver 218. Transceiver 218 optionally is used, for example, in the implementation of one or more features; for example as described in relation to transceiver 118 of FIG. 1B.

FIG. 3 is a simplified schematic of an add-on 307 coupled to an electronic device 306, according to some embodiments of the disclosure.

In some embodiments, add-on 307 includes one or more feature of add-on(s) described and/or illustrated in International Application Publication No. WO2021/224929; U.S. Provisional Patent Application No. 63/278,075 filed on Nov. 10, 2021 (attorney ref. 89431); and/or U.S. Provisional Patent Application No. 63/229,040 filed on Aug. 3, 2021 (attorney ref. 88883); the contents of all of which are incorporated herein by reference in their entirety.

In some embodiments, add-on 307 includes a portion 326 sized and/or shaped for at least partial insertion into a user's mouth. The portion 326, in some embodiments, includes dimension as described in relation to elongate element 222 of FIG. 2.

In some embodiments, portion 326 is couplable to electronic device 306, and, when coupled, modifies a FOV of one or more optical components of electronic device 306. For example, the FOV is modified according to a POV defined by the configuration and/or positioning of a portion 326. For example, the FOV may extend from a POV defined at a distal end region of portion 326. For example, where an exemplary FOV is illustrated by arrows 338. In some embodiments, transfer is of a FOV one or more imager and/or one or more illuminator.

Optionally, in some embodiments, add-on 307 includes one or more imagers, one or more illuminators, and/or one or more power supplies.

In some embodiments, add-on 307 includes a body 340. In some embodiments, portion 326 is coupled to body 340. In some embodiments, electronic device 306 is coupled to add-on 307 by body 340 e.g. by being placed into a cavity of body 340. In some embodiments, electronic device 306 includes a first face 342, and a second face 344. Optionally, first face 342 and/or second face 344 includes a screen (e.g. a touch screen). Optionally, first face 342 and/or second face 344 host one or more imager and/or one or more illuminator.

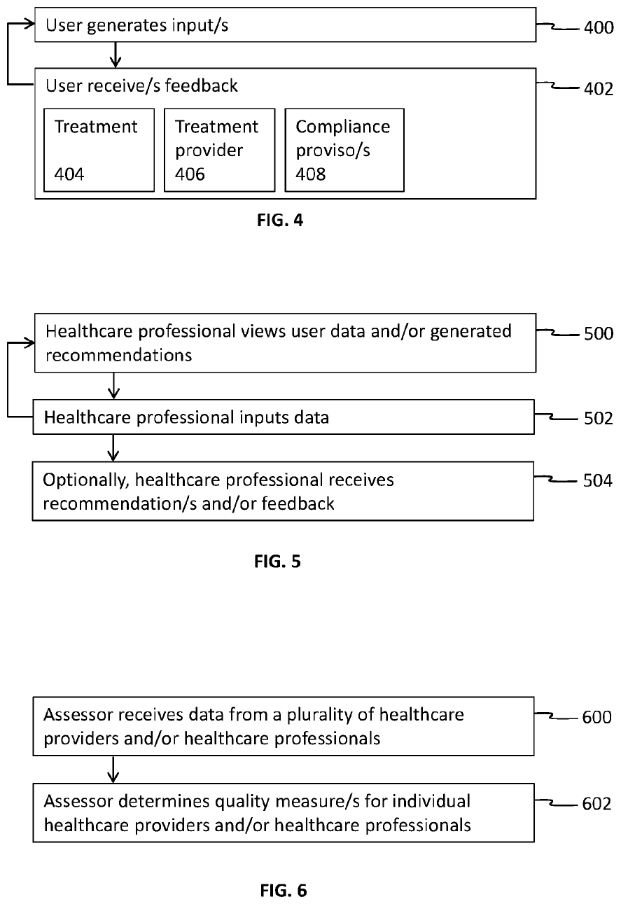

FIG. 4 is a flow chart of user experience with and/or as part of some embodiments of the disclosure.

At block 400, in some embodiments, a user generates input(s). In some embodiments, input is transmitted from the user to one or more processing applications, e.g. computing cloud-hosted applications.

In some embodiments, user-generated inputs include scan data; for example, comprising one or more user and/or care-giver scanned images of the dental features. The scanning is optionally performed in a home or other environment (e.g., care home) which is not a dental treatment office. In some embodiments, a scan is acquired during brushing; for example, using a toothbrush device including one or more features as illustrated in and/or described with respect to dental device 204 of FIG. 2. Alternatively or additionally, in some embodiments, a scan is acquired by using an electronic device (e.g. a personal electronic device). In some embodiments, before scanning, the personal electronic device is attached to an add-on including one or more features illustrated in and/or described with respect to add-on 307 of FIG. 7.

In some embodiments, inputs include information inputted into a user interface (e.g. through a person electronic device). The information includes, for example, answers to health questions. Health questions are regarding, for example, pain level(s) and/or tooth areas being scanned.

At block 402, in some embodiments, a user receives feedback, for example, through one or more user interfaces.

In some embodiments, the feedback includes a patient risk as to one or more outcomes; for example, risk of needing future treatment. Optionally, in some embodiments, risks of one more specific outcomes are displayed to a user.

In some embodiments, the feedback includes step(s) the patient can take to reduce their risk.

In some embodiments, the feedback include the raw data taken (for example the video) or the processing output (for example the 3-D model) that the user can look at and optionally share with other treatment providers to get a second opinion or cost estimation.

In some embodiments, the feedback includes details as to a treatment type 404; for example, details of a treatment protocol for home-treatment. In some embodiments, a monitoring protocol is received along with a treatment protocol e.g. for use by healthcare professional(s).

In some embodiments, feedback includes a recommendation for professional 406 to provide treatment. The recommendation, in some embodiments, is based on the treatment required, and optionally one or more additional parameters; For example, cost, efficacy of treatment, geographical location of the provider. In some embodiments, the recommendation is based on user inputted preference(s) e.g. the user supplies ranges and/or preferences for one or more of the parameters used in making the recommendation.

In some embodiments, feedback includes assessment of user progress in one or more aspects; for example, in following a treatment protocol and/or monitoring protocol, and/or in pursuing an education protocol. For example, the assessment may be that the user has improved brushing and/or knowledge of correct dental hygiene.

In some embodiments, treatment professional 406 is provided, according to an insurance company policy; e.g. according to one or more features illustrated in and/or described with respect to FIG. 8.

In some embodiments, feedback includes compliance proviso(s) 408. For example, the user is informed that if treatment, monitoring, and/or educational protocols are sufficiently complied with, the user will receive a particular insurance premium and/or a guarantee for received dental treatment(s).

In some embodiments, oral treatment feedback is “gamified”, e.g., in a form suitable for engaging children. This has potential benefits for motivating better oral healthcare. For example, in some embodiments, user feedback comprises reporting of a score of the home oral treatment (e.g., a parameter of tooth brushing or flossing such as length of time, number of strokes, and/or thoroughness of surface area coverage). Optionally, dynamics of the parameter (improvement over time, for example) are reported. Optionally, user feedback is formatted to foster competitiveness between users; which may be in the form of direct score reporting, or relative score reporting.

FIG. 5 is a flow chart of healthcare professional interaction with and/or as part of some embodiments of the disclosure.

At block 500, in some embodiments, a healthcare professional views user data. The user data, in some embodiments, includes one or more features of input(s), for example as described with respect to block 400 FIG. 4.

In some embodiments, the healthcare professional verifies user data; for example, by comparison of the user data with their previous in-person assessment of the user, and/or by comparing it to user data from a previous session.

At block 502, in some embodiments, the healthcare professional inputs data, for example, in response to data viewed. In some embodiments, for example, the healthcare professional inputs one or more of a diagnosis, a treatment feature, and a monitoring feature.

At block 504, in some embodiments (for example, based on one of both of user data and inputted healthcare professional data), the healthcare professional receives one or more recommendations, and/or feedback.

For example: in some embodiments, based on user data, automatic diagnosis and/or treatment recommendation(s) are generated, e.g. using AI analysis of the user data. Generation optionally comprises comparing the user data with previous data from the same user, from other users, and/or healthcare professional data.

FIG. 6 is a flow chart illustrating assessor use of healthcare provider and/or healthcare professional data, according to some embodiments of the disclosure.

At block 600, in some embodiments, an assessor (or other third party) receives data from a plurality of healthcare providers and/or healthcare professionals. The data include, for example, one or more of: details of user inputted data for a plurality of users, diagnoses, treatments carried out, costs, and patient dental health outcomes.

At block 602, using data received at block 600, the assessor determines one or more quality measures for one or more stakeholders. Stakeholders may include, for example, one or more of: individual healthcare providers (e.g. insurance companies), individual healthcare professionals (e.g., individual dentists), and individual healthcare professional groups (e.g. an dental service organization, optionally associated with a geographical location, through which a plurality of healthcare professionals provide services).

In some embodiments, a quality measure includes a measure of dental health state, based on from which source(s) care is received. For example, the measure takes into account and indicates, generally, whether individuals receiving healthcare from a particular stakeholder have improved dental health over time or not relative to some standard of care and/or baseline assumption. In another example, a measure is indicative of costs (e.g. over time) of dental healthcare using a specific stakeholder.

In some embodiments, a quality measure includes a measure of treatment short-term outcome (e.g., within a term of a few days or a week). In some embodiments, a quality measure includes a measure of treatment long term outcome (e.g., after a term of 1 year or more). In some embodiments, said quality measure is accumulated from data for many patients treated by a single dentist. In some embodiments, said quality measure is used for deciding on future referrals to said dentist or dental healthcare entity.

FIG. 7 is a flow chart illustrating use of user data and healthcare professional data by a third party, according to some embodiments of the disclosure.

At block 700, in some embodiments, an insurance company (and/or healthcare providing organization; the terms “insurance company”, “healthcare provider”, and “healthcare providing organization” being used interchangeably in this document) receives individual user data; e.g. including healthcare professional data for the particular user.

At block 702, in some embodiments, the insurance company receives user data for a plurality of users, and/or healthcare professional data of the users.

At block 704, in some embodiments: based on the received data (e.g. historical data over one or more time periods) for a plurality of users and for the individual user, the insurance company determines dental health risk of the individual.

At block 706, in some embodiments: optionally, the insurance company generates and/or adjusts premiums, based on dental health risk of individual(s) and/or group(s) of individuals.

At block 708, in some embodiments, the insurance company receives the individual user's monitoring data.

At block 710, in some embodiments, the insurance company adjusts the user's premium(s), based on the behavior monitored at block 708. For example, if it is determined that the user has—e.g. by their behavior (e.g. compliance), by the efficacy of their treatments, and/or by fulfilment of monitoring and/or education protocols—changed their future dental health risk, premium(s), in some embodiments, are adjusted.

In some embodiments, using data received at block 708, in some embodiments, the insurance company evaluates efficacy of treatment and/or monitoring and/or education provided to users. For example, user(s) are provided with treatment and/or monitoring and/or education plan(s) and one or more of blocks 700-708 are performed and then evaluated, based on (e.g. by comparison to) data acquired prior to implementation of the intervention plan(s).

FIG. 8 is a flow chart illustrating use of collective data, according to some embodiments of the disclosure.

At block 800, in some embodiments, user data and/or healthcare professional data (e.g. associated with the user data) for a plurality of users is received.

At block 802, in some embodiments: for a specific user, user data and/or healthcare professional data regarding the individual user is received. For example, the data comprise AI-identified potential dental healthcare issue(s), identified, e.g., from dental scan images. In other examples, the data comprise a diagnosis of a dental healthcare issue (e.g. a potential cavity) and/or a recommended treatment.

At block 804, in some embodiments, one or more recommendations and/or selections are generated for one or more of: a treatment type, a healthcare professional, and a treatment type at a specific healthcare professional. In some embodiments, recommendations and/or selections are based on the individual data received at block 802 and/or the collective data received at block 800. In some embodiments, the selection and/or recommendation is, for example, based on one or more of a determined: lowest cost, fastest recovery, and/or highest chance of recovery without relapse (e.g. for the particular treatment required).

In some embodiments, selection of a healthcare professional is based on data acquired from a plurality of user(s) and/or a plurality of healthcare professionals; e.g. over time, for example as described in relation to block 802.

In some embodiments, the healthcare professional is selected based on a particular treatment and/or other user characteristic (e.g. likelihood of compliance); for example, taking into account cost, historical proficiency and/or outcomes.

The selection may be targeted, for example, to reduce a final (total over time) cost of the treatment and/or collective costs for a plurality of users requiring this treatment. For example, an initially more expensive professional may be selected, insofar as a risk of condition recurrence and/or a need of further treatment is reduced with this professional.

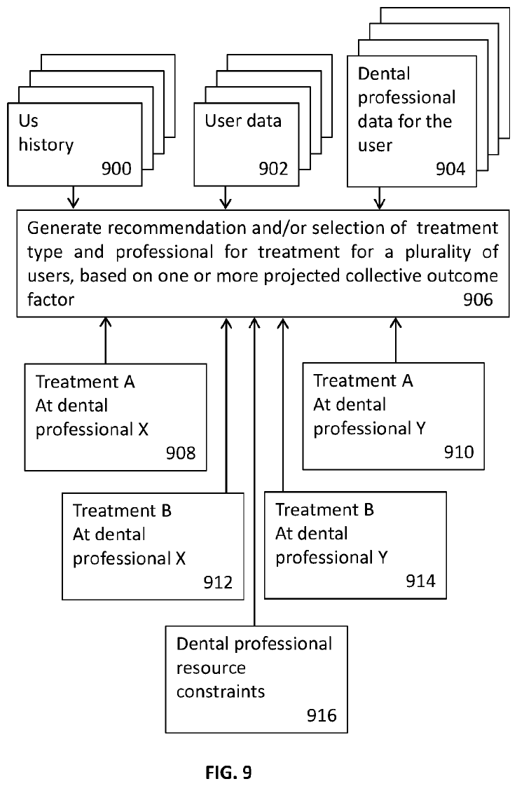

FIG. 9 is a simplified block diagram illustrating recommendation and/or selection generation, according to some embodiments of the disclosure.

In some embodiments, generator 906 determines a recommendation and/or a selection of treatment type and a professional to carry out the treatment; determinations being made for a plurality of users requiring a plurality of treatments.

For simplicity, FIG. 9 illustrates two treatment options (A and B) and two dental healthcare professionals (X and Y) but it should be understood that the method described is applicable to larger (or smaller) numbers of treatment(s) and/or healthcare professionals.

In some embodiments, generator 906 receives one or more input including for example, one or more of:
a user scan history 900 (e.g. as described elsewhere in this document);
user data 902 (e.g. as described elsewhere in this document);
dental professional data 904 (e.g. as described elsewhere in this document);
one or more features of specific treatments provided by specific dental professionals 908, 910, 912, 914,
   the one or more features include, for example: cost, likelihood of success of the treatment, aftercare requirements, and/or requirements for user participation in the treatment;
dental professional resource constraint(s) 916; for example, time available and/or abilities of the different professionals.

In assigning treatment(s) and/or healthcare providers, a potential advantage of taking into account feature(s) of specific treatments as provided by specific healthcare providers (and optionally by other relevant resources), is reduced costs to an insurance company providing the healthcare.

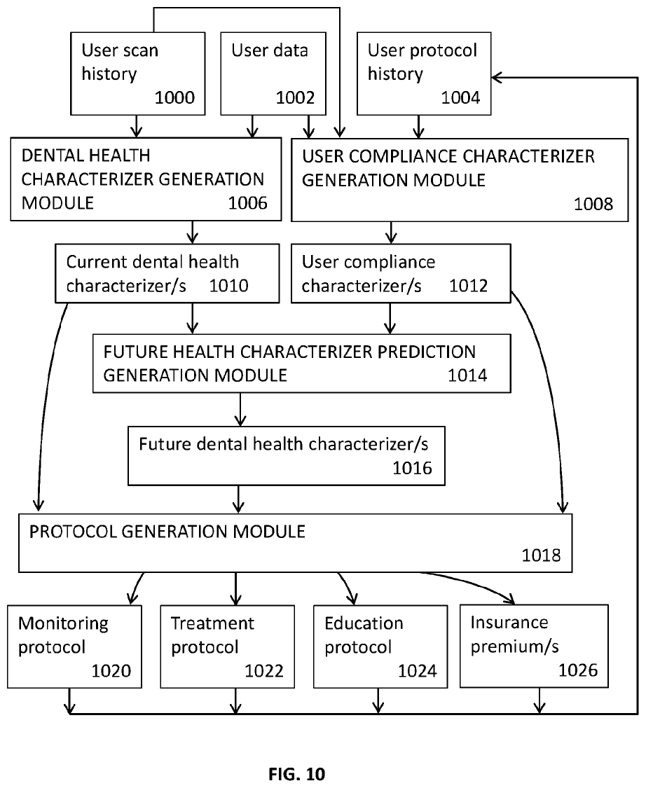

FIG. 10 is a simplified schematic block diagram illustrating protocol generation, according to some embodiments of the disclosure.

It should be understood that, in some embodiments, a portion of the block diagram illustrated is employed and other portion(s) are not. For example, each generation module should be understood as capable of being used independently.

In some embodiments, one or more of user scan history 1000, user data 1002, and user protocol history 1004 are inputs to one or more of a dental health characterizer generation module 1006 and a user compliance characterizer generation module.

In some embodiments, dental health characterizer generation module 1006 generates current dental health characterizer(s) 1010 (e.g. for each user and/or for groups of users), based on the inputs received. Optionally, dental healthcare professional data (e.g. diagnoses and/or prescribed treatments) provide an additional input, and/or are used as a part of user data 1002.

In some embodiments, a user compliance characterizer generation module generates user compliance characterizer(s) 1012 for each user, based on the inputs. Generation comprises, for example, comparison between user protocol history 1004 and user scan history 1000 for the individual. Additionally or alternatively, generation comprises using historical compliance data for other individuals, to predict the behavior of individuals. For example, an individual may be characterized as a member of a group of individuals grouped based on historical data of the individuals.

In some embodiments, a future health characterizer prediction generation module 1014 generates one or more future dental health characterizers for an individual, based on the received current dental health characterizer(s) 1010 and/or user compliance characterizer(s) 1012, for the individual user.

In some embodiments, a protocol generation module 1018 generates one or more protocols, based on one or more of: current dental health characterizer(s) 1010, future dental health characterizer(s) 1016, and user compliance characterizer(s) 1012.

In some embodiments, protocol generation includes using a lookup table. In some embodiments, protocol generation includes using a database (e.g. a best practices database). In some embodiments, a match for one or more inputs to protocol generation module 1018 is used to select from the lookup table and/or is used to find a match with the best practices database. Optionally, the lookup table selection and/or match with the best practices database are modified based on patient data; e.g. age, sex, inputted preferences, lifestyle factors (e.g. smoker, eating habits, exercise habits), likely compliance (e.g. as characterized 1010).

In some embodiments, one or more of: a monitoring protocol 1020, a treatment protocol 1022, an education protocol 1024, and insurance premium(s) 1026 become part of user protocol history 1004.

In some embodiments, monitoring protocol 1020 includes one or more of: a scan type, a scan frequency (e.g. for each scan type required), user data to be inputted, the frequency of user data input, and a frequency of evaluation of the received data (automatic and/or by a dental health professional, the frequencies of which, optionally, are different).

In some embodiments, monitoring protocol 1020 includes instructions for procedures for monitoring effectiveness of brushing. For example, the user is instructed in the use of dye to color a bio-film on dental features where scanning is used to check the brushing e.g. measuring percentage and location of effective (and/or ineffective) brushing.

In some embodiments, treatment protocol 1022 includes one or more administrations of medication (for example, topical administration to teeth and/or gums, and/or systemic administration). The protocol defines, for example, details as to the medication type, dosage, and how it is to be used (e.g. taken orally, or applied to dental features).

The medication itself includes, for example, one or more of: painkillers, antibiotic(s), anti-inflammatory medication, gum disease medication, remineralization medication. In some embodiments, treatment protocol 1022 includes instructions for brushing, flossing, replacement of braces rubber bands, replacement of clear aligners, use of whitening stripes and/or serum to get whiter teeth, and/or another home treatment.

In some embodiments, education protocol 1024 includes guidance as to brushing, flossing and/or other dental healthcare issues (e.g. lifestyle, and/or diet).

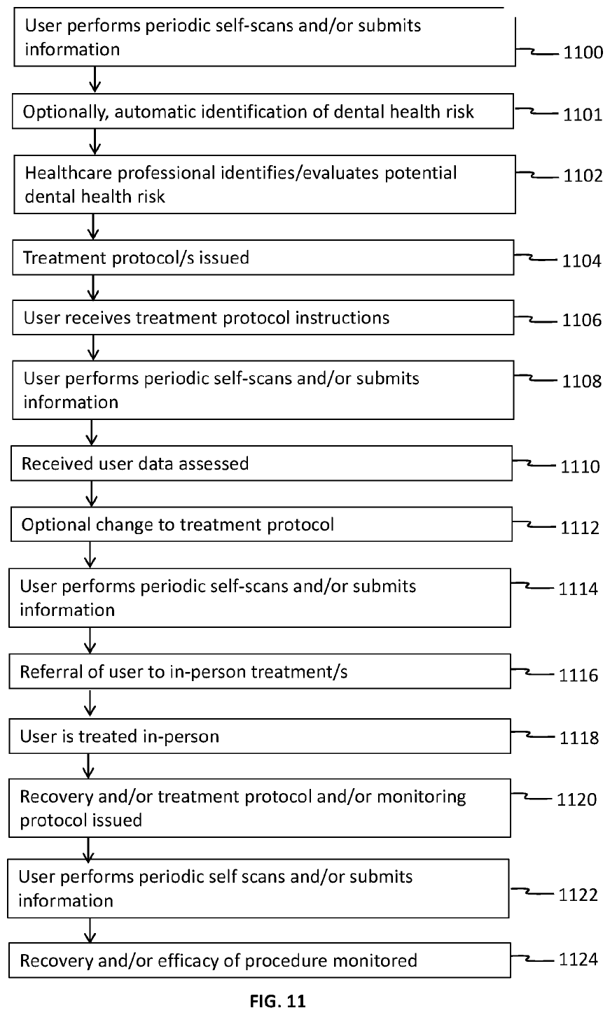

FIG. 11 is a flow chart illustrating an exemplary use case, according to some embodiments of the disclosure.

At block 1100, in some embodiments, a user performs periodic self-scans and/or submits information e.g. regarding dental health. In some embodiments, prior to starting to perform self-scans, the user is familiarized with equipment for collecting self-scans. Optionally, the user performs one or more orientation and/or education exercises to confirm their ability to acquire suitable data.

At block 1101, in some embodiments, a potential dental health risk is (optionally) automatically identified from received user scan and/or submitted information. For example, a potentially weak dental region is identified as being at risk of development of a cavity.

At block 1102, in some embodiments, a healthcare professional examining the received user's scan and/or submitted information identifies a potential dental health risk, and/or reviews a risk identified in block 1101.

In some embodiments, block 1101 is performed and not block 1102. In some embodiments, block 1102 is performed and not block 1101. In some embodiments, both of blocks 1101 are performed. In the latter case, at block 1102 the healthcare professional reviews (e.g., reviews and confirms) the automatically identified potential risk.

At block 1104, in some embodiments, one or more treatment protocols is issued. The treatment protocol is generated and/or selected optionally by the healthcare professional, and/or automatically; for example, according to inputs from the healthcare professional provided in response to viewed data.

In some embodiments, the treatment protocol includes one or more decision points. A portion of the treatment protocol performed after the decision point is determined based on a decision taken. The decision is taken by a dental healthcare professional and/or by a user, for example upon prompting via a user interface.

In some embodiments, the treatment protocol is generated automatically; for example, without interaction of a healthcare professional. For example, where block 1101 is performed (and not block 1102), the protocol is optionally generated automatically based on the automatic diagnosis at 1101.

At block 1106, in some embodiments, the user receives treatment protocol instructions. The instructions are optionally issued (and received) periodically to prompt the user to proceed with the operations of block 1108.

At block 1108, in some embodiments, the user performs periodic self-scans and/or submits information; for example, according to one or more feature as described in relation to block 1000 of FIG. 10.

At block 1110, in some embodiments, user data is assessed; for example, assessed automatically and/or by a healthcare professional. In some embodiments, the assessment forms a decision point in the treatment protocol.

At block 1112, in some embodiments, the treatment protocol is optionally adjusted e.g. based on the assessment performed at block 1110. Adjustment is performed, automatically and/or by a healthcare professional. In some embodiments, the adjustment is based on a decision received at a decision point in the treatment protocol.

At block 1114, in some embodiments, the user performs periodic self-scans and/or submits information; for example, according to one or more features as described in relation to block 1000 of FIG. 10.

At block 1116, in some embodiments, and optionally in response to another decision point and associated decision, the user is referred to an in-person treatment. For example, referral occurs upon identifying that the treatment protocol has not prevented a cavity forming, and/or that the treatment protocol and/or monitoring by the patient (e.g. performance of block 1108 and/or block 1114) has not been carried out sufficiently well.

At block 1118, in some embodiments, the user is treated in-person.

At block 1120, in some embodiments, one or more recovery and/or treatment and/or monitoring protocols are issued.

At block 1122, in some embodiments, the user performs periodic self-scans and/or submits information; for example, according to one or more feature as described in relation to block 1000.

At block 1124, in some embodiments, recovery and/or how successful the procedure has been is monitored, e.g. using the data received from block 1122.

### General

It is expected that during the life of a patent maturing from this application many relevant dental monitoring, dental treatment, toothbrush device, and electronic device technologies will be developed; the scope of the term “dental monitoring”, “dental treatment”, “toothbrush device”, and “electronic device” is intended to include all such new technologies a priori.

Thus, while various inventive embodiments have been described and illustrated herein, those of ordinary skill in the art will readily envision a variety of other means, functions, and/or structures for performing a function(s), and/or obtaining the results and/or one or more of the advantages described herein, and each of such variations and/or modifications is deemed to be within the scope of the inventive embodiments described herein. More generally, those skilled in the art will readily appreciate that any and all parameters, dimensions, materials, steps, and configurations described herein are meant to be merely an example, and actual parameters, dimensions, materials, steps, and configurations will depend upon the specific application or applications for which the inventive teachings is/are used. Those skilled in the art will recognize, or be able to ascertain using no more than routine experimentation, many equivalents to the specific inventive embodiments described herein. It is therefore to be understood that the foregoing embodiments are presented by way of example only and that, within the scope of claims supported by the subject disclosure and equivalents thereto, and inventive embodiments may be practiced otherwise than as specifically described and claimed. Inventive embodiments of the present disclosure are directed to each individual feature, device, system, article, material, kit, step, function/functionality, and method described herein. In addition, any combination of two or more such features, devices, systems, articles, materials, kits, steps, functions/functionality, and methods, if such features, systems, articles, materials, kits, steps, functions/functionality, and methods are not mutually inconsistent, is included within the inventive scope of the present disclosure, and considered embodiments.

Embodiments disclosed herein may also be combined with one or more features, as well as complete systems, devices, and/or methods, known in the art, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, as noted, various inventive concepts disclosed herein are embodied as one or more methods, of which one or more examples have been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to).

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to claims supported by the present disclosure. To the extent that section headings are used, they should not be construed as necessarily limiting. In addition, any priority document(s) of this application is/are hereby incorporated herein by reference in its/their entirety.

1. A method of dental telehealth provision comprising:
a. receiving patient self-input data, provided digitally, using a home dental camera;
b. identifying a dental health issue from said patient self-input data;
c. generating a treatment protocol including at least one activity to be carried out by the patient for treatment of said dental health issue; and
d. communicating said treatment protocol to said patient.

2. The method according to claim 1, wherein said home dental camera comprises a camera of a personal electronic device coupled to an add-on which spatially transfers a field of view of said camera through a body of said add-on.

3. The method according to claim 1, wherein said home dental camera is hosted by a toothbrush device.

4. The method according to claim 1, wherein said generating includes a dental healthcare professional specifying one or more feature of said treatment protocol.

5. The method according to claim 1, wherein said generating comprises using a look-up table and or using a best practices database.

6. (canceled)

7. The method according to claim 1, comprising adjusting said treatment protocol based on one or more of user preference and user likely compliance.

8. The method according to claim 1, wherein said treatment protocol includes instructions for one or more of:
a. using medication; and
b. dental self-treatment.

9. The method according to claim 1, wherein said self-input data comprises at least one image.

10. The method according to claim 1, wherein said self-input data comprises a plurality of images, at least two of said plurality of images including different portions of a user's mouth.

11. The method according to claim 1, wherein said self-input data comprises a plurality of images acquired during different scans.

12. The method according to claim 1, wherein said identifying comprises displaying said user self-input to a healthcare professional; and receiving inputs from said healthcare professional.

13. The method according to claim 1, wherein said identifying comprises using artificial intelligence to automatically identify said dental health issue.

14. The method according to claim 1, wherein said self-input data comprises a plurality of images acquired during different scans over a long period; and said identifying includes identifying variation in the oral cavity over said long period.

15. The method according to claim 1, wherein said identifying comprises categorizing said dental health issue; and wherein said generating comprises generating based on said categorizing.

16. The method according to claim 15, wherein said categorizing comprises categorizing said dental health issue as one or more of; acute, chronic, or a potential future dental health issue.

17. The method according to claim 1, comprising monitoring said user.

18. The method according to claim 17, wherein said monitoring comprises receiving additional patient self-input data, provided digitally, using a home dental camera.

19. The method according to claim 18, wherein said monitoring comprises communicating monitoring instructions to said user.

20. The method according to claim 18, comprising assessing outcome of said treatment protocol using said additional patient self-input data.

21. The method according to claim 20, comprising adjusting said treatment protocol based on said assessing.

22-46. (canceled)
